(19) United States
(12) Patent Application Publication
(10) Pub. No.: US 2004/0106565 A1
(43) Pub. Date: Jun. 3, 2004

(54) Gene expression, genome alteration and reporter expression in myofibroblasts and myofibroblast-like cells

(76) Inventors: Margarete Odenthal, Koln (DE); Diana Jung, Zurich (CH)

Correspondence Address: John P White, Cooper & Dunham, 1185 Avenue of the Americas, New York, NY, 10036, US

(21) Appl. No.: 10/332,733

(22) PCT Filed: Jul. 10, 2001
(86) PCT No.: PCT/EP01/07913

(30) Foreign Application Priority Data
Jul. 11, 2000 (DE) 100 33 633.7
Oct. 31, 2000 (DE) 100 53 879.7
Dec. 22, 2000 (EP) 00128446.2

(51) Int. Cl. A61K 48/00; C12N 15/85

(52) U.S. Cl. 514/44; 435/455

(57) ABSTRACT

The invention relates to specific, regulatory sequence regions of the alpha smooth muscle actin gene (a-SMA gene), and fusion structures in which said regulatory sequence regions of the a-SMA gene are operationally linked to other functional nucleic acid sequences. The invention also relates to the use of said regulatory sequence regions for cell type specific and differentiation specific reporter expression, for cell type specific and differentiation specific gene changes (alteration, mutation) and for gene expression changes in cells and organisms.

### FIELD OF THE INVENTION

The invention relates to specific, regulatory sequence regions of the alpha smooth muscle actin-gene (α-SMA gene), fusion constructs in which said regulatory sequence regions of the α-SMA gene are operatively linked to other functional nucleic acid sequences, and to the use of said regulatory sequence regions for cell type and differentiation specific reporter expression, (especially in myofibroblasts and myofibroblast-like cells), for cell type and differentiation specific gene changes, (alteration, mutation) and for gene expression changes in cells and organisms.

In detail the present invention relates to fusion constructs, in which said specific sequence region, which essentially consists of sequences that are located within the 5′-terminal region of the α-SMA gene, are operatively linked to a further functional nucleic acid sequence, preferably with peptide or protein encoding nucleic sequences, regulatory DNA-sequences or functional RNA encoding sequences; a method, wherein said fusion constructs are inserted operatively into a vector and the vector construct is introduced into eukaryotic cells; a method, wherein the cells transiently or stably transfected, transformed or infected with the reporter construct, express a reporter under control of said regulatory sequences of the α-SMA gene and the reporter expression is subsequently used to isolate or to screen embryonal or transiently α-SMA positive cells, particularly myofibroblasts or myofibroblast-like cells from a mixture of cells, a cell population, an aggregate of cells or an organism; a method, wherein by means of said fusion constructs (that contain regulatory sequences of the α-SMA gene and peptide or protein encoding and RNA encoding sequences, respectively) the gene expression respectively the cell function of α-SMA positive cells, particularly myofibroblasts and myofibroblast-like cells, in organisms or organs is manipulated by using a transgenic or knock-out/-in technology or by applying gene-therapeutic fusion vectors.

### BACKGROUND OF THE INVENTION

The α-SMA gene is a member of the actin multigene family, which encodes different isoforms of the cytoskeletal actin. The expression of the different actins is highly cell type specific and regulated in a differentiation-dependent way. In adults, the skeletal α-actin is expressed in the skeletal muscles, the cardial α-actin in the heart muscle and the α-SMA in smooth muscles. The transcriptional regulation of the α-SMA gene is achieved by an interaction of positively and negatively operating regulatory elements in the 5′-region (Blank, R. S. et al., J. Biol. Chem. 267: 984-989 (1992); Carrol, S. L. et al., J. Biol. Chem. 261: 8955-8976 (1986); Carrol, S. L. et al., Mol. Cell. Biol. 8: 241-250 (1988); Nakano, Y. et al., Gene. 99: 275-289 (1991); Min et al., J. Biol. Chem. 265: 16667-16675 (1990)). The 5′-region of the α-SMA gene contains different conserved cis-elements like two CArG-elements (CArG-A and B) (Carrol, S. L. et al., Mol. Cell. Biol. 8: 241-250 (1988) and Blank, R. S. et al., J. Biol. Chem. 267: 984-989 (1992)). The CArG motifs consist of an A/T repeat, which is flanked by CC/GG, and are currently discussed as a significant element of the muscle specific gene regulation (Chow, K. L., Schwartz, R. J., Mol. Cell. Biol., 10, 528-538 (1990) and Sobuc, K. et al., Mol. Cell. Biochem. 190:105-118 (1999)). Reporter studies in transgenic mice showed, that an additional CArG box (O) in the first intron of the α-SMA gene is necessary for an in vivo expression of α-SMA in smooth muscle cells (Mack, C. P., Owens, G. K., Circ. Res., 84: 852-861 (1999)).

The 5′-region of the α-SMA gene likewise contains two E-box consensus sequences (CA NN TG), potential binding sites for basic helix-loop-helix transcription factors (Johnson, A. D., Owens, G. K., Am. J. Physiol., 276, C1420-C1431 (1999)) and other consensus sequences (Hautmann, M. B. et al., J. Biol. Chem., 272: 10948-10956 (1997) and McNamara, C. A. et al., Am. J. Physiol., 268: C1259-C1266 (1995)), whose function is not known yet.

The α-SMA gene is expressed cell type specificly in smooth muscle cells (SMC) (Vanderkerckhove J. and Weber K., Differentiation 14: 123-133 (1979) Skalli, O. et al., J. Cell. Biol., 103: 2787-2796 (1986) and idem, J. Histochem. Cytochem., 37: 315-321 (1989)). WO00/24254 discloses a method, wherein the regulatory sequences of the 5′-region and the first intron of the α-SMA are used for the expression of heterologous genes in SMC. This publication describes also the use of these sequences within vectors for the screening for tissue remodelling, involving SMC, the possible screening for agonists and antagonists of the SMC associated tissue remodelling as well as the screening for therapeutic agents of the SMC associated tissue remodelling. In detail, WO00/24254 discloses the overall sequence of the α-SMA gene (SEQ. ID NO:1: from −2558 bp to +2784 bp of the rat α-SMA gene, i.e., the −2558 bp 5′-region and the +1 to +2784 bp region of the first exon and intron) and the sequence regions of the first intron (+773 to +1098; see SEQ ID NO:2), which is necessary for an in vivo expression in SMC.

Apart from the cell specific expression in SMC, the α-SMA gene is also transiently expressed in embryogenesis during the differentiation of skeletal and cardiac muscle cells (Ruzicka, D. L. et al., J. Cell Biol., 107: 25775-25786 (1988); Woodcock-Mitchell, J., Mitchell, J. J., Differentiation 39: 161-166 (1988)), as well as in myofibroblasts during wound healing and after myofibroblastic changes of different cell types in scaring and tissue remodeling processes after chronic organ damage (Desmouliere A. et al., Exp. Nephrol. 3: 134-139 (1995); Gabbiani G., Cardiovasc. Res. 38: 545-548 (1998); idem, Pathol. Res. Pract. 190: 851-853 (1994); idem, Am. J. Pathol. 83: 457-474 (1976)).

Myofibroblasts are cell types of mesodermal origin, which are characterized by a pronounced matrix production, a heterogenous intermediate filament pattern with desmin or vimentin and which are able to contract due to α-SMA expression (Desmouliere, A. et al., Exp. Nephrol. 3: 134-139 (1995); Gabbiani, G., Cardiovasc. Res. 38: 545-548 (1998); idem, Pathol. Res. Pract. 190: 851-853 (1994); idem, Am. J. Pathol. 83: 457-474 (1976)). They occur temporarily during wound healing, whereas persistent myofibroblasts are involved in chronic scaring processes and in the remodeling of connective tissues and organs. In different fibrotic disorders that are due to chronic organ damage like e.g. hepatitis, glomerulonephritis, myelofibrosis or fibrotic lung disorders myofbroblasts are the main matrix producers (Friedman, S. L., New. Engl. J. Med. 328: 1828-35 (1993); Gressner, A. M., Kidney Int. 49: 39-45 (1996); MacPherson, B. R. et al., Hum. Pathol., 24:710-716 (1993); Alpers, C. E. et al., Kidney Int. 41: 1134-1142 (1992); Thiele J. et al., J. Submicrosc. Cytol. Pathol. 23(1):109-21 (1991); Johnson, R. J. et al., J. Clin. Invest. 87: 847-858 (1991); Kapanci, Y. et al., Mod. Pathol. 10: 1134-42 (1997)). Therefore, myofibroblasts are also known as modulated fibroblasts; however, their progenitor cells are highly variable depending on the type of organ. Chronic kidney damage leads to a transdifferentiation of mesangial cells to myofibroblast cells (Johnson R. J. et al., J. Clin. Invest. 87: 847-858 (1991); MacPherson B. R. et al., Hum. Pathol. 24: 710-716 (1993)). In the liver chronic damage leads to liver fibrosis in which hepatic stellate cells (HSC) transdifferentiate to myofibroblasts (Friedman S. L., New. Engl. J. Med. 328: 1828-35 (1993); Gressner A. M., Kidney Int. 49: 39-45 (1996); Ramadori G. et al., Virch. Arch. [B] 59: 349-357 (1990)), regardless whether the damage was caused by a chronic alcohol abuse, viral hepatitis B or C infections or other chronic damages. Hepatic stellate cells possess pericytic properties and quiescent cells store 80% of endogenous vitamin A. (Friedman S. L., New. Engl. J. Med. 328: 1828-35 (1993)). After myofibroblastic transdifferentiation, that is characterized by matrix production, an α-SMA expression induced ability to contract, starting proliferation, vitamin A loss and a modified growth factor profile, the cells participate decisively in the maintenance and the progression of liver fibroses (Friedman, S. L., New. Engl. J. Med. 328: 1828-35 (1993); Gressner, A. M., Kidney Int. 49: 39-45 (1996)).

In bronchio-asthmatic disorders and fibrotic lung disorders myofibroblasts are the main matrix producers (Tremblay, G. M. et al., Can. Respir. J. 5(1):59-61 (1998); Gizycki, M. J. et al., Am. J. Respir. Cell. Mol. Biol. 16(6):664-73 (1997); Gabbrielli, S. et al., Pathologica.86(2):157-60) (1994)).

During progression from in situ carcinomas to invasive carcinomas the myofibroblasts emerge with their characteristic properties of induced extracellular matrix production and ability to contract (Tremblay, G. M. et al., Can. Respir. J. 5(1):59-61 (1998); Gizycki, M. J. et al., Am. J. Respir. Cell. Mol. Biol. 16(6):664-73 (1997); Gabbrielli, S. et al., Pathologica.86(2):157-60) (1994)).

In a lot of carcinomas the stroma cells are myofibroblasts (Terada, T. et al., J. Hepatol. 24: 706-12(1996); Faouzi, S. et al., J. Hepatol. 30(2): 275-84 (1999); Kosmehl, H. et al., Br. J. Cancer. 81(6): 1071-9 (1999); Pujuguet, P. et al., Am. J. Pathol. 148: 579-92 (1996); Chomette, G. et al., Pathol. Res. Pract. 186(1): 70-9 (1990); Ronnov-Jessen, L. et al., J. Clin. Invest. 95(2): 859-73 (1995)), that influence tumor growth in a paracrine dependent manner (Neaud, V. et al., Hepatology 26: 1458-66 (1997); Tomlinson J. et al., Clin. Cancer Res. 5(11): 3516-22 (1999). But the tumor cells themselves can be mybrofibroblastic descendants as in e.g. the myofibroblastoma and the myofibrosarcoma of different organs as well (Gocht, A. et al., Pathol. Res Pract. 195(1): 1-10 (1999);Unden, A. B. et al., J. Invest. Dermatol. 107 (2): 147-53 (1996); Auger, M. et al., Arch. Pathol. Lab. Med. 113(11): 1231-5 (1989)).

Gene therapeutic approaches for chronic fibrotic disorders and tumors: Since myofibroblasts are the central cell types in many sclerosing diseases and tumors, they are the ideal target cells for therapy, including gene-therapy. By cell type specific, and differentiation-dependent control of the expression

(a) of genes encoding e.g. intracellular signal mediators (Walther, W., Stein, U., Mol. Biotechnol. 13(l): 21-28 (1999)) or

(b) RNA encoding sequence regions, which influence the transcription or translation of specific genes by binding (Bai, J. et al., Mol. Ther. 1(3): 244-254 (2000)) or

(c) of an enzyme for cyclic recombination (CRE), that modifies the floxp-flanked genetic regions (Sauer, B. Methods 14: 381-392 (2998)), which allows the therapy of specific target cells. Often viral vesicles like adenoviral or retroviral derivatives (Miller, N. Whelan, J. Hum. Gene Ther. 8, 803-815; Mountain, A., Tibtech. 18: 119-128 (2000); Schiedner, G. et al., Nat. Genet. 18, 180-183 (1998); Parr, M. J., Nat. Med. 3, 1145-1149 (1997); Ory, D. S. et al., Proc. Natl. Acad. Sci. 93:11400-11406 (1996); Feng, M. et al., Nat. Biotechnol. 15: 866-870 (1997)) are used as vectors. But also the DNA of a plasmid construct can be administered either alone or in an aggregate with liposomes or polymeric bodies (Hengge, U. et al., Nat. Genet. 10:161-166 (1995); Gottschalk, S. et al., Gene Ther. 3:3410-3414 (1990); Wagner, E. et al., Proc. Natl. Acad. Sci. 87:3410-3414 (1990)). For gene-therapeutic approaches and modifications of the myofibroblastic cell function, suitable regulatory sequences or promoters are necessary, which are able to direct the appropriate changes in cell function and expression in myofibroblasts, a requirement that is not fulfilled yet. Only by using regulatory sequences within the promoter, which direct the properties of the function of the cell according to myofibroblasts (a) by an altered gene expression, (b) by modulating protein synthesis or (c) by genome alteration, the cells might respond to gene therapy.

### SUMMARY OF THE INVENTION

During characterization of the regulating sequence region of the α-SMA gene (subsequently called α-SMA-5′ region), it was surprisingly found that single regions within, namely within −698 to +18 of the α-SMA-5′ sequence region (numbering, if not stated otherwise, is based upon the α-SMA gene from rattus norvegicus as shown in FIG. 7A) show activities, which differ from the activities of the whole region. Thus, it was found that the region from −189 to +18 of the α-SMA gene is activating transcription, whereas the region from −698 to −190 shows cell type specific and differentiation dependent regulatory properties, i.e. it is responsible for the cell type specific control together with the Core-region (−189 to +18). Based on the finding that certain regions can control gene expression and also function in myofibroblasts, myofibroblast-like cells and embryonal, transiently or induced SMA-expressing cells, a system for myofibroblastic control was developed. The present invention therefore relates to

(1) a nucleic acid sequence comprising

(i) one or more functional regions from the regulatory sequence regions of the alpha Smooth Muscle Actin Gene (α-SMA gene) and

(ii) at least one additional functional nucleic acid sequence, that is operatively linked with sequence (i), wherein the regulatory sequence region preferably consists of nucleotides −698 to +18 of the α-SMA gene (the numbering refers to the sequence from rattus norvegicus as shown in FIGS. 5A and 7A), and wherein the additional functional nucleic acid sequence does not encode for the α-SMA gene;

(2) a preferred embodiment of the nucleic acid sequence as defined in (1), wherein the regulatory sequence region is derived from rat and the nucleic acid sequence preferably comprises one or more functional regions of the sequence shown in FIG. 5A (SEQ ID NO:1), especially of the sequence shown in FIG. 7A (SEQ ID NO:5);

(3) a preferred embodiment of the nucleic acid sequence as defined in (2), wherein the functional region is a transcription activating nucleic acid sequence and particularly derived from the sequence −189 to +18 of the sequence shown in FIG. 7A (i.e. from the sequence 7C; SEQ ID NO:7);

(4) a preferred embodiment of the nucleic acid sequence as defined in (2), wherein the functional region is a cell type specific nucleic acid sequence and particularly derived from sequence −698 to −190, and particularly preferred derived from sequence −698 to −215 as defined in FIG. 7A;

(5) a preferred embodiment of the nucleic acid sequence (as defined in (1), wherein the regulatory sequence region is derived from the mouse and the nucleic acid sequence preferably shows one or more functional regions of the sequence shown in FIG. 5B (SEQ ID NO:2), especially from region SEQ ID NO:18;

(6) a preferred embodiment of the nucleic acid sequence as defined in (5), wherein the functional region is

(a) a transcription activating nucleic acid sequence and derived from nucleotides 490 to 697 of the sequence shown in SEQ ID NO:18 and/or

(b) a cell type specific nucleic acid sequence and particularly derived from nucleotide 1 to 489 of the sequence shown in SEQ ID NO:18;

(7) a preferred embodiment of the nucleic acid sequence as defined in (1), wherein the regulatory sequence region is derived from human and the nucleic acid sequence preferably comprises one or more functional regions of the sequence shown in FIG. 5C, particularly (SEQ ID NO:3) from the sequence shown in SEQ ID NO:19;

(8) the preferred embodiment of the nucleic acid sequence as defined in (7), wherein the functional region

(a) is a transcription activating nucleic acid sequence and particularly derived from nucleotides 513 to 715 of the sequence shown in SEQ ID NO:19 and/or

(b) is a cell type specific nucleic acid sequence and particularly derived from nucleotides 1 to 513 of the sequence shown in SEQ ID NO:19;

(9) a preferred embodiment of the nucleic acid sequence as defined in (1), wherein the regulatory sequence region is derived from chicken and the nucleic acid sequence preferably comprises one or more functional regions of the sequence shown in FIG. 3D, particularly of SEQ ID NO:20;

(10) a preferred embodiment of the nucleic acid sequence as defined in (9), wherein the functional region

(a) is a transcription activating nucleic acid sequence and particularly derived from the nucleotides 497 to 699 of the sequence shown in SEQ ID NO:20 and/or

(b) is a cell type specific nucleic acid sequence and particularly derived from nucleotides 1 to 496 of the sequence shown in SEQ ID NO:20;

(11) a transcription activating nucleic acid sequence as defined in (3), (6), (8) or (10);

(12) a cell type specific nucleic acid sequence as defined in (4), (6), (8) or 10;

(13) a vector containing a nucleic acid sequence as defined in (11) and/or (12);

(14) a vector into which a nucleic acid sequence as defined in (1) to (10) is inserted;

(15) eukaryotic cells or organisms that are transiently or stably transfected, transformed or infected with a nucleic acid sequence as defined in (1) to (10) or with a vector as defined in (13) or (14),

(16) a method for generation of eukaryotic cells as defined in (15) comprising the transfection, transformation or infection of parental cells with the nucleic acid sequences as defined in (1) to (10) or with vectors as defined in (13) or (14);

(17) a method for the generation of organisms as defined in (15), comprising the injection of ES-cells, which are transfected with nucleic acid sequences as defined in (1) to (10) or with vectors as defined in (13) or (14) into blastocysts of the organisms and the implantation into a foster mother;

(18) a method for the isolation or for the screening of smooth muscle cells, myofibroblasts or myofibroblast-like cells from a mixture of cells, a cell population, a cell aggregate or an organism, comprising the expression of a reporter gene in a eukaryotic cell or an organism as defined in (15);

(19) a method for the manipulation of gene expression and/or cell function of smooth muscle cells or myofibroblasts in organisms and/or organs, comprising the introduction of nucleic acid sequences as defined in (1) to (10) or a vector as defined in (14) into the organs or organisms;

(20) a pharmaceutical composition containing a nucleic acid sequence as defined in (1) to (10), (11) or (12), or a vector as defined in (13) or (14) and

(21) the use of a functional region out of the regulatory sequence region of the α-SMA gene as defined before under (1) to (12) for the generation of a pharmaceutical composition for cell type and differentiation specific reporter expression, for cell type and differentiation specific gene alteration and for manipulation of gene expression and/or cell function, particularly in embryonal cells, transiently positive for SMA, in myofibroblasts and in myofibroblast-like cells.

In the following the invention is exemplified by means of the accompanying figures and examples.

### DESCRIPTION OF THE FIGURES

FIG. 1: Measurement of luciferase reporter activity in resting and myofibroblastic activated hepatic stellate cells (HSC) under control of the PRL-700-reporter construct.

FIG. 2a: Immunocytology of single cells, cultivated after their dissociation from differentiated EB. In pSMA-GFP-190 transfected cells even α-SMA negative cells (1C) showed a GFP-expression (1B). In pSMA-GFP-700 transfected cells the GFP-expression (2B) was limited to α-SMA expressing cells (2C).

FIG. 2b: Design of the SMA-GFP-700 and SMA-GFP-190 reporter vector for stable transfection in embryonal stem cells or transgenic mouse models. As a heterologous regulatory sequence the first intron of the chicken β-actin is cloned into the constructs between the GFP-reporter (EGFP-variant) and the 716 bp (SEQ ID NO:5) or the 207 bp (SEQ ID NO:7) of the 5′-region of the α-SMA gene. For the selection of stable transfectants the construct carries not only an ampicillin resistance but also resistance against neomycin, which allows G418 selection in eukaryotic cells.

FIG. 3a: Luciferase reporter activities of SMA-constructs of different length (SMA-125 to SMA-700; see FIG. 7) measured after transient transfection of embryonal myotubes and myoblasts of the cell line L6. L6-cells, which differentiate into α-SMA expressing myotubes after serum depletion, showed a 4- to 5-fold induction of all constructs compared to the precursor L6-myoblasts. The SMA-190 construct is strongly transactivating, whereas the SMA-700 construct controls a differentiation dependent reporter expression. The activity of the SMA-constructs were normalized to SV40 promoter activity (100%). Three independent assays in duplicates were done.

FIG. 3b: Plasmid constructs for the expression of a peptide or polypeptide of interest under control of the 716 bp-5′-sequence (SEQ ID NO:5) of the α-SMA gene. For the detection of expression the open reading frame (ORF) can be supplemented by one or more tags added in frame such as the coding sequence for streptavidin (SEQ ID NO:34) and/or an epitope like myc (SEQ ID NO:35) that is recognized by an antibody (9E10) and/or a sequence encoding 1 to 5 histidines (SEQ ID NO:36) that allow purification. The sequence of the peptide as well as the linked tags are under the control of the cell type specific and differentiation dependent regulatory sequence region of the α-SMA gene.

FIG. 4: LoxP-mediated Cre-recombination under control of the α-SMA-5′-gene region:

a.: The transcriptional control of the Cre-gene (SEQ ID NO:32) by the 5′-region of the α-SMA-5′-sequence region (SEQ ID NO:5) in combination with the first intron of the β-actin (SEQ ID NO:31) leads to expression of the Cre-gene in myofibroblasts after myofibroblastic differentiation (1), the cre-mediated excision of the loxP flanked neomycin-gene (neo) (2) and the subsequent expression of the gene of interest (3).

b.: The transcriptional control of the Cre-gene (SEQ ID NO:32) by the 5′-region of the α-SMA-5′-sequence region (SEQ ID NO:5) in combination with the first intron of the β-actin (SEQ ID NO:31) leads to expression of the Cre-gene in myofibroblasts after myofibroblastic differentiation (1), the cre-mediated excision of the loxP-flanked gene of interest (2), so that a subsequent expression of the gene of interest is interrupted (3).

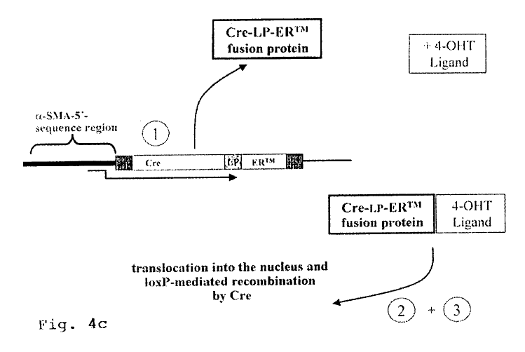

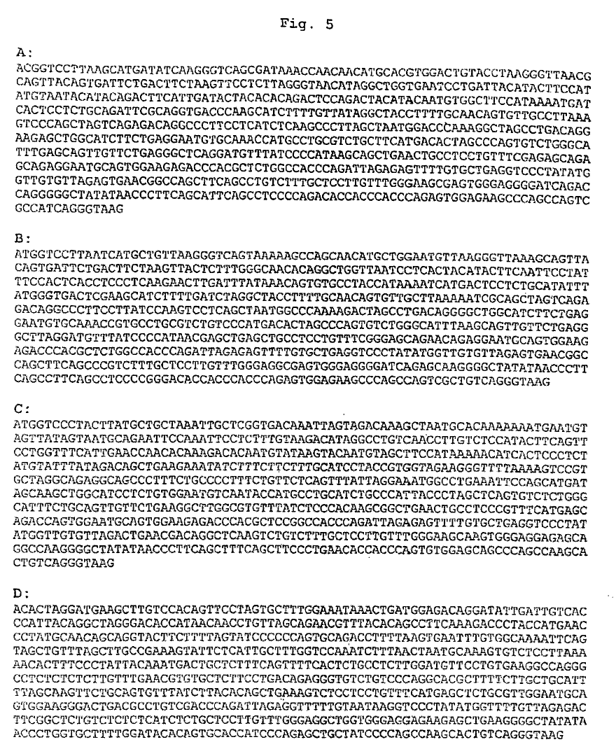

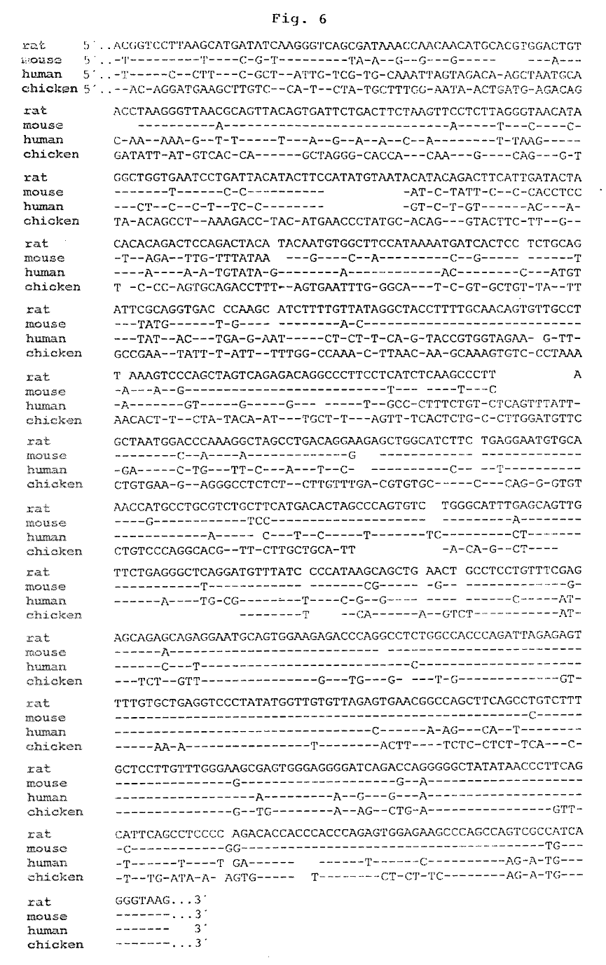

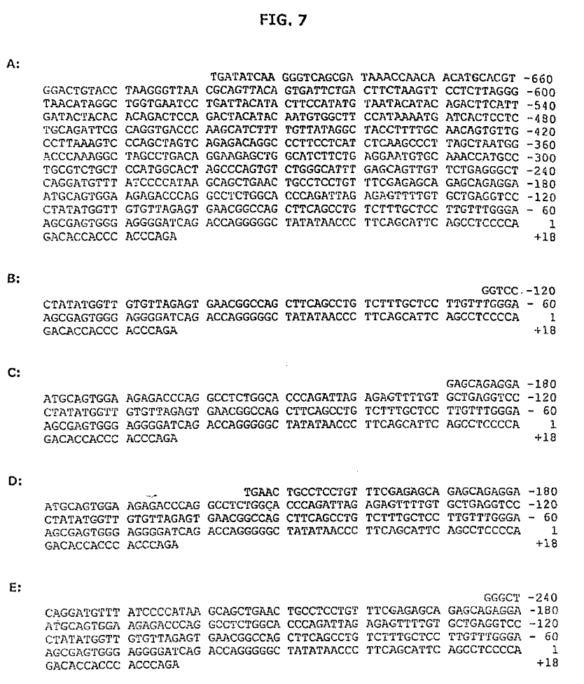

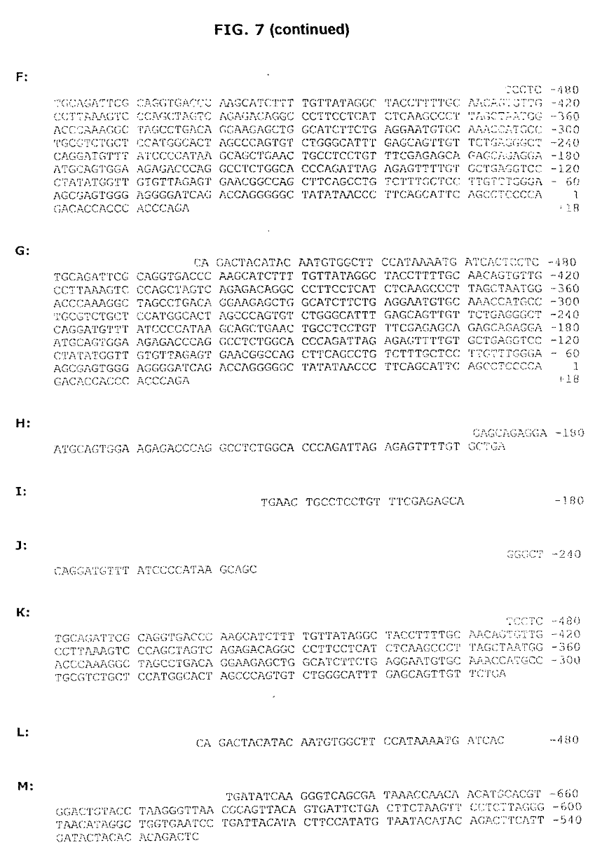

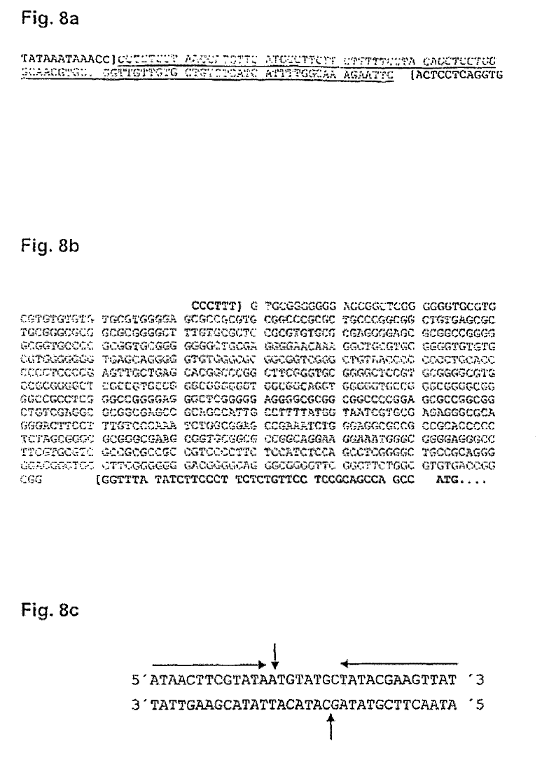

c.: The transcriptional control of the Cre-ER-fusion-gene (SEQ ID NO:32+ER™, Danielian PS et al., Mol. Endocrinol. 7: 232-240, 1993) by the 5′-region of the α-SMA-5′-sequence region (SEQ ID NO:5) in combination with the first intron of the β-actin (SEQ ID NO:31) leads to expression of the Cre-ER-fusion-gene in myofibroblasts after myofibroblastic differentiation (1). After adding or injecting of 0.1 to 2 mg 4-OHT, the ER-peptide can bind to the ligand and a Cre-ER/4-OHT-complex is formed. The complex is subsequently translocated into the nucleus and the Cre-mediated steps (2 and 3) as shown in FIGS. 4a and b take place.

nS: nuclear signal peptide sequence; hatched box: intron sequence; LP: Linker peptide sequence; ER™: sequence for an altered estrogen receptor with high affinity for the synthetic ligand 4-hydroxytamoxifen (4-OHT), that does not bind endogenous 17-β-estadriol; β-A: β-actin-intron (SEQ ID NO:31); and 4-OHT: 4-hydroxytamoxifen

FIG. 5: 5′-upstream region and beginning of exon1 of α-SMA of different species (region −713 to +52, numbering based upon sequence A)

A: rat (Rattus norvegicus; SEQ ID NO:1; Acc. No S76011; Blank, R. S. et al., J. Biol. Chem. 267(2): 984-989 (1992)).

B: mouse (Mus musculus; SEQ ID NO:2; Acc. Nos. M57409 M35194; Min, B. H. et al., J. Biol. Chem. 265: 16667-16675 (1990)).

C: human (Homo sapiens; SEQ ID NO:3; Acc. No. J05193; Reddy, S. et al., J. Biol. Chem. 265(3): 1683-1687 (1990)).

D: chicken (Gallus gallus; SEQ ID NO:4; Acc. M13756 D00041 N00041; Carroll, S. L. et al., J. Biol. Chem. 261(19): 8965-8976 (1986)).

FIG. 6: Alignment of the sequences shown in FIG. 5.

FIG. 7: Shows the preferred α-SMA-sequence of the present invention, derived from rat (Rattus norvegicus) (SEQ ID NO:)

A: 5′-region of the α-SMA gene: −698 to +18 (5)

B: 5′-region of the α-SMA gene: −124 to +18 (6)

C: 5′-region of the α-SMA gene: −189 to +18 (7)

D: 5′-region of the α-SMA gene: −214 to +18 (8)

E: 5′-region of the α-SMA gene: −244 to +18 (9)

F: 5′-region of the α-SMA gene: −484 to +18 (10)

G: 5′-region of the α-SMA gene: −521 to +18 (11)

H: 5′-region of the cα-SMA gene: −189 to −125 (12)

I: 5′-region of the α-SMA gene: −214 to −190 (13)

J: 5′-region of the α-SMA gene: −244 to −215 (14)

K: 5′-region of the α-SMA gene: −484 to −245 (15)

L: 5′-region of the α-SMA gene: −521 to −485 (16)

M: 5′-region of the α-SMA gene: −698 to −522 (17)

FIG. 8A: β-1-globin gene of Oryctolagus cuniculus; intron II/exon III-transition site; Acc.No: V00882: sequence 1250-1343 (SEQ ID NO:30)

FIG. 8B: Cytoplasmic β-actin gene from Gallus gallus; Acc.No: X00182, intron I (544-1542); (SEQ ID NO:31) shown in the sequence 550 bp to 1503 bp.

FIG. 8C: Layout of a floxp-site: The FloxP-sequence (SEQ ID NO:33) consists of 13 bp inverted repeats (horizontal arrows), that flank an 8 bp asymmetric Core-region, which is cut at two sites by Cre (vertical arrows).

### DETAILED DESCRIPTION OF THE INVENTION

The present invention is based on the surprising finding that specific regulatory sequences of the α-SMA gene, particularly those which contain one or two of the sequences shown in FIG. 7A to M (SEQ ID NOs:5 to 17), are able to control the gene expression of operatively linked functional nucleic acid sequences, e.g. those of a reporter gene or of another functional gene, in myofibroblasts or myofibroblast-like cells.

The nucleic acid sequence of embodiment (1) of the invention contains one or more functional regions out of the regulatory sequence region of the α-SMA gene. This regulatory sequence region can be derived from mammals, particularly from primates, rodents, or birds, and particularly preferred from rat, mouse, human or chicken (Blank, R. S. et al., J. Biol. Chem. 267(2): 984-989 (1992); Min, B. H. et al., J. Biol. Chem. 265: 16667-16675 (1990); Reddy, S. et al., J. Biol. Chem. 265 (3): 1683-1687 (1990); Carroll, S. L. et al., J. Biol. Chem. 261 (19): 8965-8976 (1986)).

In the preferred embodiment (2) the regulatory sequence region is derived from rat (Blank, R. S. et al., J. Biol. Chem. 267: 984-989 (1992)), as mentioned above. Particularly preferred is therein, that the functional region contains a transcription activating nucleic acid sequence and particularly preferably is derived from sequences shown in FIG. 7C. Hereby, those transcription activating sequences are preferred, which have at least 10, preferably at least 40 and particularly preferred at least 60 consecutive bases from the sequence shown in FIG. 7C and particularly preferred comprise the sequence shown in FIGS. 7B and C shown sequences (SEQ ID NOs:6 or 7). Alternatively or additionally the functional region can be a cell type specific or differentiation dependent regulated nucleic acid sequence, preferably one that is derived from sequence −698 to −190 of FIG. 7A (nucleotide 1 to 509 from SEQ ID NO:5), particularly preferred from sequence −698 to −215 shown FIG. 7A. Hereby those cell type specific sequences are preferred that have at least 25, preferably at least 35 consecutive bases from sequence −698 to −190 of FIG. 7A and particularly preferred comprise those sequences shown in FIG. 7I or 7M.

According to the preferred embodiments (5)-(10) the regulatory sequence region is derived alternatively from mouse, human or chicken (Min, B. H. et al., J. Biol. Chem. 265: 16667-16675 (1990); Reddy, S. et al., J. Biol. Chem. 265 (3): 1683-1687 (1990); Carroll, S. L. et al., J. Biol. Chem. 261 (19): 8965-8976 (1986)), wherein the functional region are defined as mentioned before under (5) to (10). Particularly preferred in accordance with the present invention is hereto, that the transcription activating nucleic acid sequence comprises at least 10, preferably at least 40 and particularly preferred at least 60 consecutive bases out of the transcription activating sequences described under (6), (8) or (10) and at least 25, preferably at least 35 consecutive bases out of the cell type specific sequences defined in (6), (8) or (10), respectively. Particularly preferred are those partial sequences that are described in SEQ ID NOs:18, 19 and 20, which correspond to sequences 7A to M of the rat (according to the alignment in FIG. 6).

Another functional nucleic acid sequence according to embodiment (1) of the invention can be a peptide or protein encoding DNA sequence (including but not limited to a reporter genes, sequences that encode pharmacologically active proteins and regulatory DNA-sequences) or a functional RNA encoding sequence (including but not limited to a ribozyme).

Previous studies have shown the linkage of α-SMA gene fragments with reporter genes (inter alia Nakano, Y. et al., Gene 99(2): 285-9 (1991); Blank, R. S. et al., J. Biol. Chem. 15, 267(2): 984-9 (1992); Johnson, A. D. und Owens, G. K., Am. J. Physiol. 276: C1420-31 (1999); Hautmann, M. B. et al., J Biol. Chem. 272(16): 10948-56, (1997); Simonson, M. S. et al., Am. J. Physiol. 268(4 Pt 2): F760-9 (1995); Kim, J. H. et al., Biochem. Biophys. Res. Commun. 190(3): 1115-21 (1993); McNamara, C. A. et al., Am. J. Physiol. 268: C1259-66 (1995) as well as WO0/24254 and U.S. Pat. No. 5,885,769). Suitable reporter genes for a myofibroblastic control are e.g. the genes listed in table 1. Other functional genes according to the invention are e.g. genes that encode for pharmacologically active proteins or peptides, and regulatory DNA or RNA sequences.

Table 1: Examples for possible reporter genes, which expression can be controlled by the α-SMA-5′-sequence

| | | sequence | |
|---|---|---|---|
| reporter gene | detection | (SEQ ID NO) | reference |
| destabilized | 488-507 nm | 21 | EP-A-0892047 |
| EG-FP red | 538-583 nm | 22 | |
| fluorescent | | | |
| protein | | | |
| GFP and | | | |
| variants | 380-440 nm | 23 | EP-A-0892047 |
| e.g. blue | | | |
| variant | | | |
| renilla | luminescence | 24 | |
| luciferase | | | |
| firely | | 27 | |
| luciferase | | | |
| SEAP¹ | BCIP², NBT³ | 26 | |
| CAT⁴ | | 28 | |
| hGH⁵ | | 29 | JP 1985234584 |
| β-galactosidase | x-Gal⁷ | 25 | |
| horseradish | DAB⁶, AEC⁷ | | EP-A-0234075 |
| peroxidase | | | |

¹SEAP: secreted alkaline phosphatase
²BCIP: 5-Bromo-4-chloro-3-Indolylphosphate
³NBT: Nitroblue-Tetrazolium
⁴CAT: Chloramphenicol-Acetyltransferase
⁵hGH: human growth hormone
⁶DAB: 3.3′-Diaminobenzidin
⁷AEC: 3-Amino-9-Ethylcarbazol

The vector according to embodiment (13) comprises conventional transfection vectors and vectors suitable for genetic transfer.

The eukaryotic cells or organisms according to embodiment (15) of the present invention, can be produced using methods for transfection, transformation or infection of parental cells and infection of ES-cells in blastocysts of organisms and their implantation into a foster mother, respectively, well known to the person skilled in the art.

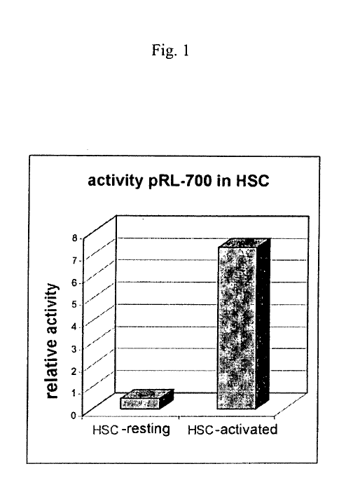

Up to now, it was not possible to screen for myofibroblastic activation by using a reporter, since a regulatory sequence region, which is specific for the transition from resting cells to myofibroblastic cells, had not been found. By fusion of a sequence region from −698 to +18 (SEQ ID NO:5) with a reporter gene (see table 1) and insertion into a transfer vector and subsequent transfection screening for a myofibroblastic activation is possible (FIG. 1). In previous methods of screening for myofibroblastic activation immunocytological or immunohistological procedures were chosen, which comprise several steps like fixation of cells, antigen blocking, antibody incubation and detection (F. Hofman, Current protocols in immunology, Chapt. 5.8: Immunohistochemistry; Eds. J. E. Coligan et al., National Institute of Health, USA (1996)). Usually the immunocytological or immunohistological detection of myofibroblasts was done by immunochemical staining of the α-SMA protein. Quantitative measurements of the activation e.g. by a toxicological reagent, however, were not possible this way. In the presented invention luminating reporter proteins were preferably chosen to act as α-SMA-5′-sequence controlled reporter of myofibroblastic transformation and the activation of the cells was measured in an luminometer (MLX Microplate Luminometer of Dynex Technologies, Chantilly, USA) in a quantitative and highly sensitive manner (FIG. 1). With a quantitative reporter assay, quantitative toxicological and pharmacological studies are feasable by the method for the first time, which allow an estimation of the myofibroblastic activation potential of the substance and thereby detection of a possible potential for chronic relevant organ damage.

Assaying the myofibroblastic transition of cells, which contain the vector with the α-SMA-5′-sequence reporter fusion construct, holds the potential of screening for therapeutic pharmaceutical compositions; here the reduction of myofibroblastic activity, e.g. reduced by a toxin or a fibrogenic growth factor like e.g. transforming growth factor beta (TGF β), can be read out by means of the α-SMA-5′-sequence controlled reporter.

The method presented according to the invention, wherein the α-SMA-5′ sequence regions (−698 to +18) are used for reporter control comprises apart from screening for myofibroblastic activity the isolation of certain cells by means of the α-SMA-5′ controlled reporter expression. In the presented method, this is preferably done by operative linkage of α-SMA-5′ sequence regions with a fluorescent reporter (e.g. GFP see table 1), so that after introducing the vector construct into an organism or a cell population, transient embryonal SMA positive cells or myofibroblastic induced cells can be isolated from a mixture or assembly of cells by a FACS-sorter (FACS: fluorescence activated cell sorting) by means of fluorescence. Alternatively, the linkage of the α-SMA-5′ sequence region with other reporters permits the isolation of myofibroblasts and areas with myofibroblast-like cells by microdissection under light or fluorescence microscopic control.

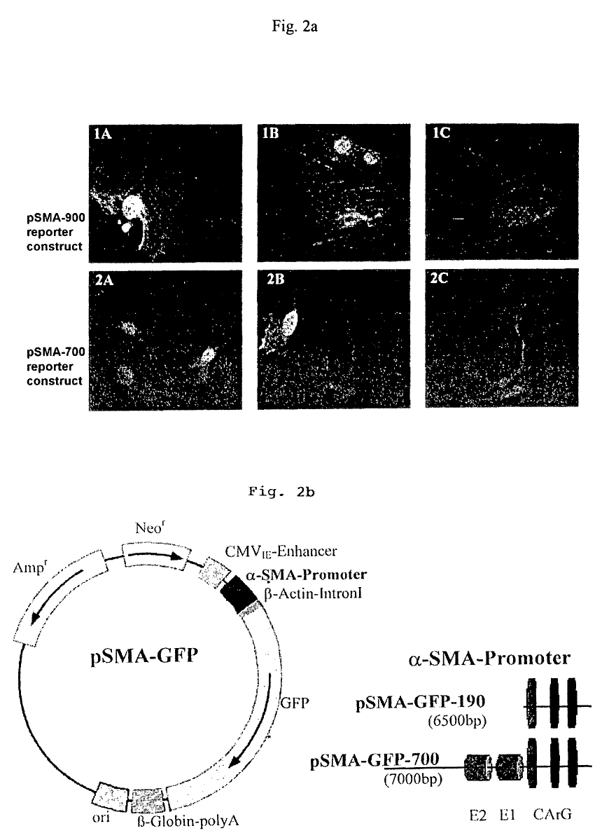

The presented invention also comprises the role of the sequence region −698 to +18 of the α-SMA gene for the specific gene expression in embryonic stem cells (ES), which are able to differentiate to embryonal cardiomyocytes, smooth muscle cells or skeletal muscle cells in embryoid bodies (Evans, M. J., Kaufmann, M. H., Nature, 292, 154-156 (1981)). The comparison of stably into ES transfected constructs with different domains of the α-SMA-5′ located sequence regions showed, that for a selection of cardiomyocytes, skeletal muscle cells and smooth muscle cells from embryonal cell aggregates a reporter control by the sequence region of −189 to +18 (FIG. 7C; SMA-190) is not sufficient, but that additional 5′ located domains within the region of −698 to −190 of the α-SMA gene (Seq. I, J, K, L and M of FIG. 7) are necessary (FIG. 2).

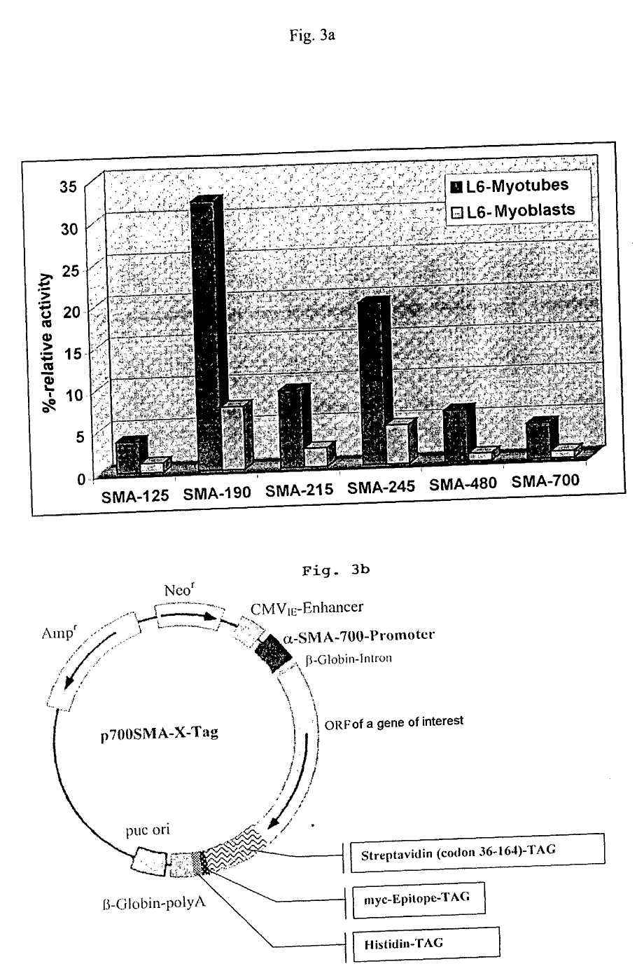

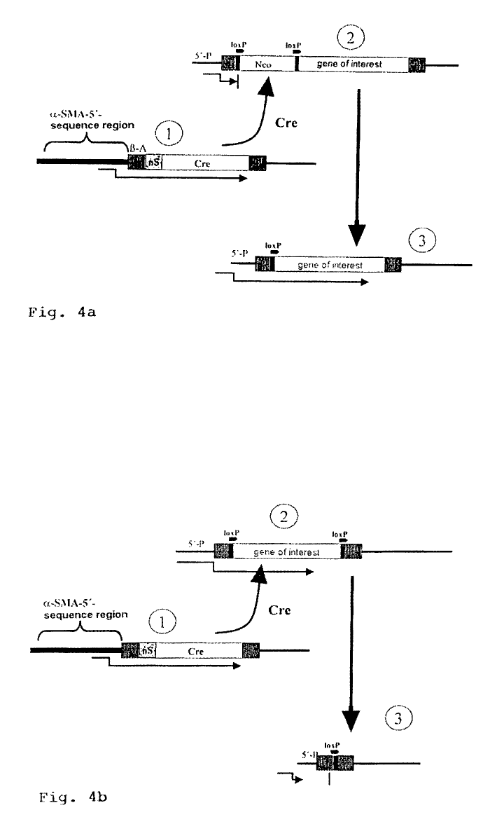

The invention likewise relates to the operative linkage of the α-SMA-5′ sequence region or partial regions of the sequence (Seq. A to M in FIG. 7) with other nucleic acid sequences, which influence expression. Examples are the intron sequences of the β-globin or the chicken β-actin gene (see FIGS. 8A and B, SEQ ID NO:30 and 31, respectively), but also other enhancer sequences like the viral CMV as in the vector shown in FIG. 3. But also other not mentioned expression influencing sequences can be operatively linked to the α-SMA-5′-sequences or domains thereof (sequence A to M of FIG. 7).

If not a reporter gene but another peptide or protein encoding genetic region is linked to the α-SMA-5′ sequence region (SEQ ID NOs:5 to 17), this leads to an α-SMA-5′ controlled expression of the operatively linked genetic region. In a preferred embodiment of the invention, sequence A of FIG. 7 is used for control of the expression of the genetic region of interest. FIG. 1 or FIG. 3 show the differentiation depending expression of the transcriptional control of sequence A of FIG. 7: The transcriptional control by sequence A inhibits the expression in myoblasts or resting myofibroblastic precursors, whereas after myotube differentiation or myofibroblastic transdifferentiation it leads to the expression of the linked genetic region. If sequence C of FIG. 7 is linked to the target gene, it leads to an expression in many cell types like e.g. in fibroblasts, the linkage of sequences A, F, G of FIG. 7 leads to an expression in myofibroblastic cells or SMC-descendants. The genetic region transcriptionally controlled by α-SMA-5′ sequence regions (Seq. A to M of FIG. 7) can in turn be linked with other additional sequence domains. The linkage, while having regard to the reading frame, of sequences encoding certain tags or reporter domains (FIG. 3b) allows the monitoring of gene expression or the purification of proteins or peptides encoded by the genetic region transcriptionally controlled by α-SMA sequences.

By operatively linking the α-SMA-5′ sequence region (Seq. A to M from FIG. 7) with sequences, that contain RNA encoding DNA or genetic regions for signal mediators, one can interfere functionally with the phenotype of myofibroblastic cells. After insertion of such constructs into a vector that is suitable for genetic therapy (as e.g. different generations of adenoviral, retroviral or lentiviral vectors and their derivates (Ory, D. S. et al., Proc. Natl. Acad. Sci. 93: 11400-11406 (1996); Feng, M. et al., Nat. Biotechnol. 15: 866-870 (1997)) and plasmid vectors, respectively, either naked or as aggregates in combination with charged polymers or liposomes (Hengge, U. et al., Nat. Genet. 10: 161-166 (1995); Gottschalk, S. et al., Gene Ther. 3: 448-457 (1996)) a prerequisition for gene-therapeutic approaches of myofibroblastic cells or myofibroblastic-like cells is met, which can be used in sclerosing disorders (Desmouliere, A. et al., Exp. Nephrol. 3: 134-139 (1995); Gabbiani, G., Cardiovasc. Res. 38: 545-548 (1998); idem, Pathol. Res. Pract. 190: 851-853; idem Am. J. Pathol. 83: 457-474 (1976)) or tumor disorders (Terada, T et al., J. Hepatol. 24: 706-12(1996); Faouzi, S. et al., J. Hepatol. 30(2): 275-84 (1999); Kosmehl, H. et al., Br. J Cancer. 81(6): 1071-9 (1999); Pujuguet, P. et al., Am. J. Pathol. 148: 579-92 (1996); Chomette, G. et al., Pathol. Res. Pract. 186(1): 70-9 (1990); Ronnov-Jessen, L. et al, J. Clin. Invest. 95(2): 859-73 (1995); Gocht, A et al., Pathol. Res. Pract. 195(1): 1-10 (1999)).

The operative linkage of regions from sequence A to M of FIG. 7, wherein in the present invention sequence A of FIG. 7 was preferably used, with the enzyme for cyclic recombination (Cre) (SEQ ID NO:32) allows new recombination of genetic regions, that were flanked by a floxP-sequence (FIG. 8C, SEQ ID NO:33) in myofibroblastic differentiated cells. Depending on the function of the Cre-enzyme and of the orientation of the floxP-sequences an inversion, an excision, integration or translocation of the floxed genetic regions under control of the selected regulating α-SMA-5′ sequence region can occur. Selected possible constructs of the vector in the present invention are shown in FIG. 4.

In the method (17) according to the present invention transfected ES-cells are injected into blastocysts of a host organism and are implanted into a foster mother (V. Papaioannou, R. Johnson, Gene Targeting. A Practical Approach, ed. A. L: Joyner, Oxford UK (1993); T. Doetschman, Transgenis Animal Technology. A Laboratory Handbook, Academic Press Inc. San Diego, USA (1994)). After back crossing the transgenic chimeric animals that were born and grown to adults, homozygous animals were injected with the toxin to be tested and then screened for transgenic gene expression.

Alternatively, in method (17) constructs without a vector backbone can be introduced into the prenucleus by microinjection and thereby lead to a transgenic organism (J. W. Gordon et al., Methods Enzymol. 101: 411-33 (1983); B. Hogan et al., Manipulating the mouse embryo. A laboratory manual, Gold Spring Harbor, N.Y. (1994)).

Organisms according to embodiments (15) and (17) of the present invention comprise mammals including, but not limited to primates, rodents and ungulates (wherein mammals only concern non-human organisms, if human organisms are not patentable according to the relevant national or regional laws). The eukaryotic cells according to embodiment (15) comprise all possible cells and cell lines from the above mentioned mammals.

The “pharmaceutical compositions” according to embodiments (20) and (21) of the present invention (it can be also a diagnostica depending on task, application, kind of the additional functional nucleic acid sequence etc.) can—depending on the application form—contain apart from the nucleic acid sequences/vectors of the present invention also commercial additives, diluents and the like. The pharmaceutical compositions herein are suitable for the manipulation of gene expression and/or cell function of myofibroblasts or myofibroblast-like cells, which means they can be used for the treatment and diagnosis of a multitude of disorders (as elaborated on in the background of the invention) including fibrotic diseases caused by chronic organ damage (like e.g. hepatitis, glomerulonephrits, myelofibrosis or fibrotic lung disorders) and tumors.

The invention is illustrated using the following, non-limiting examples.

### EXAMPLES

Materials and Methods

TABLE 2
Used primers

| | | region of the |
|---|---|---|
| primer | sequence (SEQ. ID. NO.) | α-SMA gene |
| SMA-710-F | 5′-GTC CTT AAG CTT GAT ATC AAG GGT CAG CG (37) | −710 to −682 |
| SMA-30R | 5′-GGC TTC TGA ATT CTG GGT GGG TGG TGT CT (38) | +1 to +29 |
| SMA-245F | 5′-TGT TCT CAG GCT CAG GAT G (39) | −252 to −235 |
| SMA-190F | 5′-CTG TTT CGA GAG AGC AAA GCT TAG G (40) | −203 to −181 |
| SMA-100 | 5′-GAG TGA ACG GCC AGC TTC (41) | around −100 |

The following commercially available vectors were used during this study:

As common cloning vectors pBluescript-SKII+ (Clontech, Heidelberg) and pGEM3Z (Promega, Mannheim) were used. Regions of the α-SMA promoter were subcloned into the promoterless luciferase vector pRL-null (Promega, Mannheim). To be able to compare the activity of the generated construct additionally other vectors of the pRL-family were used like pRL-SV40 and pRL-CMV as well as those of the pGL3 family (Promega, Heidelberg).

### Example 1

### Generation of the α-SMA-5′-Sequence

From isolated rat DNA a 736 bp long fragment from the 5′-promoter-region of the α-SMA gene was amplified and cloned. Initially this was done by a PCR amplification of the region from −708 to +28 from genomic rat DNA using primer SMA-710F (HindIII) and SMA-30R (EcoRI), which contain the mentioned restriction sites within their sequence. After restrictions the resulting fragment (−698 to +18) was inserted by ligation into pRL-null and the resulting vector, pRL-SMA-700 for the experiments in eukaryotic cells, was propagated in an E. coli K12 strain (see sequence of FIG. 7A).

### Example 2

### Screening for Myofibroblastic Differentiation by Reporter Expression Under Control of a 5′-Region of the α-SMA Gene

A. Preparation of the α-SMA-5′ sequence region regulated reporter vectors: For the preparation of the α-SMA-5′ region reporter constructs different α-SMA-5′ regions of the α-SMA were inserted into reporter vectors. The amplified product of primers SMA-710-F and SMA-30-R from genomic rat DNA (see above) was restricted with EcoRI and HindIII, cloned into pBSSK+ (Stratagene) and thereby pB-SMA-700 was generated. Using the restriction sites in the α-SMA-5′ region of the α-SMA gene the fragments SMA-480 (PstI), SMA-215 (PvuII) and SMA-125 (DraII) were generated, by excising them from pB-SMA-700 with EcoRI and a specific restriction enzyme and first subcloning them into pGEM (Promega) or pB-SK (Stratagen). Thereby, the following plasmids were generated: pGEM-SMA-480, pGEM-SMA-215 and pB-SMA-125. Using the primer SMA-245F (PstI) or SMA-190F (HindIII) as sense primer and SMA-30R as antisense primer specific fragments were generated, which were cloned subsequently via their primer associated restriction sites, thereby producing pGEM-SMA-245 and pB-SMA 190.

All promoter fragments of the α-SMA promoter were subcloned via EcoRI/HindIII or EcoRI/SacI into pRL-null, whereby the 6 deletion constructs SMA-700 to SMA-125 of different length were produced.

B. Transient transfection: Transfections were done using the transfection reagent FuGENE™6 (Roche Molecular Biochemicals, Mannheim). 1.5 μg of plasmid DNA were applied per experiment, composed of 1.2 μg of pRL-700 and 0.3 μg of pGL3-control plasmid, corresponding to a DNA reagent ratio of 1:3. For the transfection resting hepatic stellate cells (HSC), which had been isolated from rat liver by enzymatic liver perfusion and density gradient centrifugation (Knock, D. S., de Leeuw, A. M., Exp. Cell. Res., 139, 468 471 (1982)) and differentiated HSC, which were differentiated into myofibroblasts after culture on plastic dishes (Falcon) (Geerts, A. et al., J. Hepatology, 9, 59-68 (1989)) were employed.

C. Determination of reporter activity under control of sequence A in resting HSC and in myofibroblastic HSC: Activity was measured with a dual-luciferase reporter assay system (DLR, Promega, Mannheim). In transient transfections only myofibroblastic cells showed a clear activity of the pRL-700 reporter construct. α-SMA negative cells showed reporter activity that was below 1% of relative activity. In myofibroblasts the reporter was induced by a factor of 14. Resting cells had no activity (>1%) (FIG. 1). Therefore, the construct can be used for the screening for myofibroblastic differentiation.

### Example 3

### Reporter Under Control of an α-SMA-5′-Sequence in Stably Transfected Organisms and Cells

A. Construction of a reporter vector under control of α-SMA: From a modified GFP-expression vector (pCAGGS) (Ikawa, M. et al., Develop. Growth Differ. 37: 455-459 (1995)) with a fragment of the chicken-β-actin-promoter and the first intron of the chicken-β-actin (SEQ ID NO:31) the cardial promoter element was excised by restriction digest with SnaBI and ApaI and replaced with the respective α-SMA-5′ sequence regions (SEQ ID NO:5 or SEQ ID NO:7) after digest of pRL-700 or pRL-190 with XhoI and EcoRI. After purification of the bands by an agrose gel the protruding ends were filled up, to enable “Blunt End”-cloning. In addition, to inhibit the religation of the vectors, they were dephosphorylated at the 5′-ends. After a ligation reaction and transformation in E. coli positive clones were isolated, their plasmid DNA purified and sequenced to verify the insertion of the α-SMA-5′ sequence region in the correct orientation. Sequencing was done using the labelled primer SMA-100 (TM=63° C.), which primes in the region −100 of the α-SMA-promoter. The constructs are shown in FIG. 2b.

B. Screening and isolation of stable transfectants using the α-SMA-5′-sequence (sequences of FIGS. 7A and 7C) controlled reporter activity: To generate stable ES-transfectants the two GFP-reporter constructs pSMA-GFP-700 and -190 (FIG. 2b) were made and transfected into embryonal murine D3-stem cells. Stably transfected clones were selected using a neomycin (G418)-resistance that is contained in the GFP-construct. For this purpose 50 μg/ml neomycin (10 mg/ml, GibcoBRL, Eggenstein) was added 24 hours after transfection.

To allow differentiation of stem cells these were cultivated without feeder cells and without addition of LIF in DMEM (440 mg D-glucose, 110 mg natriumpyruvate, L-glutamine, Sigma) with 20% FCS (GibcoBRL, Gaithersburg, Md.), 1×non-essential amino acids (GibcoBRL), 1×penicillin-streptomycin (GibcoBRL) and 0.1 mM β-mercaptoethanol (Sigma). For the generation of embryoid bodies (EB) 2.25×10⁵ and 4.25×10⁵ cells, respectively, were diluted into 1 ml of culture medium. 20 μl of the respective cell suspension (450 or 800 cells) were cultivated as hanging drops (Drab, M. et al., FASEB, 11: 905-911 (1997) und Kolossov, E. et al., J. Cell. Biol., 143: 2045-2056 (1998)).

Immunohistology in EB that were transfected with the PSMA-GFP-190 promoter fragment, showed that GFP positive cell areas did not always express α-SMA and that not all α-SMA positive cells expressed GFP. GFP positive cells could be assigned to actin expressing cardial or skeletal muscle cell types. Caldesmon positive smooth muscle cells showed no GFP expression.

In contrast, the pSMA-GFP-700 construct showed a specific GFP expression that could be demonstrated in cardial and skeletal regions as well is in caldesmon positive smooth muscle cells. The observed reporter pattern under control of sequence A of FIG. 5 of the α-SMA gene corresponds in differentiated ES-cells to that during in embryogenesis. The SMA-700 fragment (SEQ ID NO:5) is therefore a functional control sequence for embryonal and transiently SMA positive cells.

By means of reporter activity cells could be decollated after trypsinization (FIG. 2a) and analyzed separately.

### Example 4

### Generation of Mice with Transgenic Gene Expression in Myofibroblastic Cells

A. Stable transfection of the constructs shown in FIGS. 2b/3 into ES-cells: For the generation of stable transfected embryonal stem cells the α-SMA transgene vectors were linearized by a restriction digest using a site within the ampicillin gene and dissolved in water after purification (1 μg/μl). Afterwards an electroporation was performed. For this purpose 5×10⁶ ES-cells were resuspended into 0.7 ml PBS and transferred into a cuvette. 30 μg of linearized plasmid DNA in a volume of 100 μl were used per electroporation. After addition of DNA into the cuvette an incubation on ice for 10 min. took place, to allow the formation of precomplexes. Thereafter, an electroporator from Biorad (Munich) was used to produce an impulse of 500 pF and 0.24 V. Thereafter cells were incubated for 20 min. on ice to allow regeneration and subsequently seeded onto neomycin resistant feeder cells.

The ES-cells were injected into blastocysts of a host mouse and implanted into the foster mother (V. Papaioannou, R. Johnson, in Gene Targeting. A Practical Approach, ed. A. L: Joyner, Oxford UK (1993); T. Doetschman, Transgenis Animal Technology. A Laboratory Handbook, Academic Press In.c San Diego, USA (1994)). Transgenic chimaeras that had been born and grown to adults were backcrossed and the resulting homozygous animals were injected with toxin and screened for transgenic gene expression.

Alternatively, constructs without vector backbone (see FIG. 2b) can be introduced into the prenucleus by microinjection and thereby lead to transgenic mice (J. W. Gordon et al., Methods Enzymol. 101: 411-33 (1983); B. Hogan et al., in Manipulating the mouse embryo. A laboratory manual, Gold Spring Harbor, N.Y. (1994)).

B. Chronic Liver Damage by Toxin Injection and Myofibroblastic Screening

To induce chronic liver damage mice were interperitoneally injected two weekly with 4% carbon tetra chloride in mineral oil (6 ml/kg body weight) for 2, 4, 6 or 8 weeks. The mice were examined for pulmonitis and nephritis as well as for the occurrence of myofibroblastic cells in the lung or kidney. Since carbon tetra chloride leads mainly to hepatitis, the activation of myofibroblastic cells was observed in the livers of transgenic GFP-mice.

1. Use of a nucleic acid sequence comprising
(i) one or more functional regions of the regulatory sequence region of the alpha-smooth muscle actin gene (α-SMA gene) that consists of nucleotides −698 to +18 of the α-SMA gene, wherein the numbering refers to the sequence of rattus norvegicus as shown in FIGS. 5A and 7A, and
(ii) at least one additional functional nucleic acid sequence that does not encode for the natural α-SMA and that is operatively linked with sequence (i)
for the preparation of a pharmaceutical composition for manipulation of gene expression and/or cell function of transient or after activation, SMA-positive cells, of myofibroblasts or myofibroblast-like cells.

2. Use of claim 1, wherein the nucleic acid sequence of the regulatory sequence region of the α-SMA gene
has at least 10, preferably at least 20 consecutive bases from nucleotides −698 to +18 of the α-SMA gene and/or
is derived from mammals, particularly from primates, rodents or birds and particularly preferred from rat, mouse, human or chicken.

3. Use of claim 2, wherein the regulatory sequence region is derived from rat and the nucleic acid sequence comprises preferably one or more functional regions of the sequence shown in FIG. 5A (SEQ ID NO:1), particularly of the sequence shown in FIG. 7A (SEQ ID NO:5).

4. Use of claim 3, wherein the functional region
(i) is a transcription activating nucleic acid and particularly derived from the sequence shown in FIG. 7C (SEQ ID NO:7), and wherein preferably the transcription activation sequence has at least 10, preferably at least 40 and particularly preferred at least 60 consecutive bases of the sequence shown in FIG. 7C and particularly preferred comprises the sequences shown in FIG. 7B or 7C (SEQ ID NOs:6 or 7); or
(ii) is a cell type specific nucleic acid sequence and particularly derived from sequence −698 to −190 of FIG. 7A (nucleotides 1 to 509 of SEQ ID NO:5), particularly preferred from sequence −698 to −215 of FIG. 7A (nucleotides 1 to 484 of SEQ ID NO:5) or from the sequence shown in FIG. 7I (SEQ ID NO:13), and wherein preferably the cell specific sequence has at least 25, preferably at least 35 consecutive bases from sequence −698 to −190 of FIG. 7A and particularly preferred comprises the sequences shown in FIG. 7I or 7M (SEQ ID NO:13 or 17).

5. Use of claim 3 or 4, wherein the nucleic acid sequence comprises a transcription activating as well as a cell type specific nucleic acid sequence and/or has one of the sequences shown in SEQ ID NOs:5 to 17.

6. Use of claim 2, wherein the regulatory sequence region is derived from mouse and the nucleic acid sequence preferably has one or more of the functional regions of the sequence shown in FIG. 5B (SEQ ID NO:2), particularly of the sequence shown in SEQ ID NO:18.

7. Use of claim 6, wherein the functional region
(a) is a transcription activating nucleic acid sequence and particularly derived from nucleotides 490 to 697 of SEQ ID NO:18 and/or
(b) is a cell type specific nucleic acid sequence and particularly derived from nucleotides 1 to 498 of the sequence shown in SEQ ID NO:18.

8. Use of claim 2, wherein the regulatory sequence region is derived from human and the nucleic acid sequence preferably has one or more functional regions of the sequence shown in FIG. 5C (SEQ ID NO:3), particularly of the sequence shown in SEQ ID NO:19.

9. Use of claim 8, wherein the functional region
(a) is a transcription activating nucleic acid sequence and particularly derived from nucleotides 513 to 715 of the sequence shown in SEQ ID NO:19 and/or
(b) is a cell type specific nucleic acid sequence and particularly derived from nucleotides 1 to 512 of the sequence shown in SEQ ID NO:19.

10. Use of claim 2, wherein the regulatory sequence region is derived from chicken and the nucleic acid sequence has preferably has one or more functional regions of the sequence shown in FIG. 5D (SEQ ID NO:4), particularly of the sequence shown in SEQ ID NO:20.

11. Use of claim 10, wherein the functional region
(a) is a transcription activating nucleic acid sequence and particularly derived from nucleotides 497 to 699 of the sequence shown in SEQ ID NO:20 and/or
(b) is a cell type specific nucleic acid sequence and particularly derived from nucleotides 1 to 496 of the sequence shown in SEQ ID NO:20.

12. Use of any one claim of claims 1 to 11, wherein the additional functional nucleic acid sequence is a peptide or protein encoding DNA sequence and particularly chosen from reporter genes, sequences that encode pharmacologically active proteins, and regulatory DNA sequences, or encodes a functional RNA, particularly a ribozyme.

13. A method for the manipulation of gene expression and/or cell function of myofibroblasts or myofibroblast-like cells in organisms and organs, comprising the introduction of nucleic acid sequences as defined in claims 1 to 12, or of vectors wherein said nucleic acid sequences are inserted into the organisms or organs.

14. A method of claim 13, which is an in vivo or in vitro method and wherein particularly
(i) the insertion into the organisms or organs is done by using transgenic knock-out/-in technology, or
(ii) the method is an application of a gene-therapeutic fusion vector.

15. A nucleic acid sequence, comprising
(i) one or more functional regions of the regulatory sequence region of the alpha-smooth muscle actin gene (α-SMA gene) that consists of nucleotides −698 to +18 of the α-SMA gene, wherein the numbering refers to the sequence from rattus norvegicus as shown in FIGS. 5A and 7A, and
(ii) at least one additional functional nucleic acid sequence that is operatively linked with sequence (i) and does not encode a natural α-SMA and is selected from DNA sequences that encode pharmacologically active proteins, regulatory sequences or functional RNA encoding sequences.

16. A nucleic acid sequence of claim 15 as defined in claims 2 to 11.

17. Transcription activating nucleic acid sequences or cell type specific nucleic acid sequences as defined in claims 4, 7, 9, or 11.

18. A vector comprising a nucleic acid sequence as defined in claim 17, or into which a nucleic acid sequence as defined in claims 15 or 16 is inserted.

19. Eukaryotic cells or organisms, which are transiently or stably transfected, transformed or infected with a nucleic acid sequence as defined in claim 15, 16 or 17 or with a vector as defined in claim 18, and that preferably
(i) express a reporter under control of the regulatory sequences of the α-SMA gene; or
(ii) express or contain a pharmalogically active protein, a regulatory DNA sequence or functional RNA sequence under control of the α-SMA gene.

20. A method for the generation of eukaryotic cells as defined in claim 19 comprising the transfection, transformation or infection of parental cells with nucleic acid sequences as defined in claims 15, 16 or 17, or with a vector as defined in claim 18.

21. A method for the generation of organisms as defined in claim 19 comprising the injection of ES-cells with the nucleic acid sequences as defined in claims 15, 16 or 17, or with vectors as defined in claim 18, into blastocysts of organisms and their implantation into a foster mother.

22. A method for the manipulation of gene expression and/or the cell function of smooth muscle cells or myofibroblasts in organisms or organs comprising the insertion of nucleic acid sequences as defined in claims 15, 16 or 17, or of vectors as defined in claim 18 into the organisms or organs,
(i) wherein the insertion into the organisms or organs is preferably done by using transgenic knock-out/-in technologies, or
(ii) wherein the method is preferably an application of gene-therapeutic fusion vectors.

23. A pharmaceutical composition containing a nucleic acid sequence as defined in claims 1 to 12, 15, 16 or 17, or a vector as defined in claim 18, wherein the pharmaceutical composition preferably is suited for the manipulation of gene expression and/or cell function of smooth muscle cells or myofibroblasts in organisms or organs.

24. Use of a functional region from the regulatory sequence region of the α-SMA gene for the myofibroblastic and differentiation dependent reporter expression or for the myofibroblastic and differentiation dependent gene manipulation, wherein preferably the functional region of the α-SMA gene
(i) is a nucleic acid sequence as defined in claim 17;
(ii) is a nucleic acid sequence as defined in claims 1 to 12 or 15 to 16; and/or
(iii) is present in a vector as defined in claim 18.

25. A method for the isolation of or screening for transiently or after activation SMA-positive cells, of myofibroblasts or myofibroblast-like cells from a mixture of cells, a cell population, a cell aggregate or an organisms, comprising
the expression of a reporter gene in a eukaryotic cell or organism, with the reporter under the control of the regulatory sequence of the α-SMA gene as defined in claims 1 to 12 or 15 to 16; and
the detection of the reporter.

26. Use of the regulatory sequences of the α-SMA gene as defined in claims 1 to 12 or 15 to 16 for the generation of a diagnostics for the isolation or screening of transiently or, after activation SMA-positive cells of myofibroblasts or of myofibroblast-like cells from a mixture of cells, a cell population, a cell aggregate or an organism.


SEQUENCE LISTING (41 sequences)

SEQ ID NO: 1 - DNA, 764 bp, Rattus norvegicus
acggtccttaagcatgatatcaagggtcagcgataaaccaacaacatgcacgtggactgtacctaagggttaacgcagttacagtgattctgacttctaagttcctcttagggtaacataggctggtgaatcctgattacatacttccatatgtaatacatacagacttcattgatactacacacagactccagactacatacaatgtggcttccataaaatgatcactcctctgcagattcgcaggtgacccaagcatcttttgttataggctaccttttgcaacagtgttgccttaaagtcccagctagtcagagacaggcccttcctcatctcaagcccttagctaatggacccaaaggctagcctgacaggaagagctggcatcttctgaggaatgtgcaaaccatgcctgcgtctgcttcatgacactagcccagtgtctgggcatttgagcagttgttctgagggctcaggatgtttatccccataagcagctgaactgcctcctgtttcgagagcagagcagaggaatgcagtggaagagacccacgctctggccacccagattagagagttttgtgctgaggtccctatatggttgtgttagagtgaacggccagcttcagcctgtctttgctccttgtttgggaagcgagtgggaggggatcagaccagggggctatataacccttcagcattcagcctccccagacaccacccacccagagtggagaagcccagccagtcgccatcagggtaag

SEQ ID NO: 2 - DNA, 745 bp, Mus musculus
atggtccttaatcatgctgttaagggtcagtaaaaagccagcaacatgctggaatgttaagggttaaagcagttacagtgattctgacttctaagttactctttgggcaacacaggctggttaatcctcactacatacttcaattcctatttccactcacctccctcaagaacttgatttataaacagtgtgcctaccataaaatcatgactcctctgcatatttatgggtgactcgaagcatcttttgatctaggctaccttttgcaacagtgttgcttaaaaatcgcagctagtcagagacaggcccttccttatccaagtcctcagctaatggcccaaaagactagcctgacaggggctggcatcttctgaggaatgtgcaaaccgtgcctgcgtctgtcccatgacactagcccagtgtctgggcatttaagcagttgttctgagggcttaggatgtttatccccataacgagctgagctgcctcctgtttcgggagcagaacagaggaatgcagtggaagagacccacgctctggccacccagattagagagttttgtgctgaggtccctatatggttgtgttagagtgaacggccagcttcagcccgtctttgctccttgtttgggaggcgagtgggaggggatcagagcaaggggctatataacccttcagccttcagcctccccgggacaccacccacccagagtggagaagcccagccagtcgctgtcagggtaag

SEQ ID NO: 3 - DNA, 763 bp, Homo sapiens
atggtccctacttatgctgctaaattgctcggtgacaaattagtagacaaagctaatgcacaaaaaaatgaatgtagttatagtaatgcagaattccaaattcctctttgtaagacataggcctgtcaaccttgtctccatacttcagttcctggtttcattgaaccaacacaaagacacaatgtataagtacaatgtagcttccataaaaacatcactccctctatgtatttatagacagctgaagaaatatctttcttctttgcatcctaccgtggtagaagggttttaaaagtccgtgctaggcagaggcagccctttctgcccctttctgttctcagtttattaggaaatggcctgaaattccagcatgatagcaagctggcatcctctgtggaatgtcaataccatgcctgcatctgcccattaccctagctcagtgtctctgggcatttctgcagttgttctgaaggcttggcgtgtttatctcccacaagcggctgaactgcctcccgtttcatgagcagaccagtggaatgcagtggaagagacccacgctccggccacccagattagagagttttgtgctgaggtccctatatggttgtgttagactgaacgacaggctcaagtctgtctttgctccttgtttgggaagcaagtgggaggagagcaggccaaggggctatataacccttcagctttcagcttccctgaacaccacccagtgtggagcagcccagccaagcactgtcagggtaag

SEQ ID NO: 4 - DNA, 747 bp, Gallus gallus
acactaggatgaagcttgtccacagttcctagtgctttggaaataaactgatggagacaggatattgattgtcacccattacaggctagggacaccataacaacctgttagcagaacgtttacacagccttcaaagaccctaccatgaaccctatgcaacagcaggtacttcttttagtatccccccagtgcagaccttttaagtgaatttgtggcaaaattcagtagctgtttagcttgccgaaagtattctcattgctttggtccaaatctttaactaatgcaaagtgtctccttaaaaacactttccctattacaaatgactgctctttcagttttcactctgcctcttggatgttcctgtgaaggccagggcctctctctcttgtttgaacgtgtgctcttcctgacagagggtgtctgtcccaggcacgcttttcttgctgcattttagcaagttctgcagtgtttatcttacacagctgaaagtctcctcctgtttcatgagctctgcgttggaatgcagtggaagggactgacgcctgtcgacccagattagaggtttttgtaataaggtccctatatggttttgttagagacttcggctctgtctctctcatctctgctccttgtttgggaggctggtgggaggagaagagctgaaggggctatataaccctggtgcttttggatacacagtgcaccatcccagagctgctatccccagccaagcactgtcagggtaag

SEQ ID NO: 5 - DNA, 716 bp, Rattus norvegicus
tgatatcaagggtcagcgataaaccaacaacatgcacgtggactgtacctaagggttaacgcagttacagtgattctgacttctaagttcctcttagggtaacataggctggtgaatcctgattacatacttccatatgtaatacatacagacttcattgatactacacacagactccagactacatacaatgtggcttccataaaatgatcactcctctgcagattcgcaggtgacccaagcatcttttgttataggctaccttttgcaacagtgttgccttaaagtcccagctagtcagagacaggcccttcctcatctcaagcccttagctaatggacccaaaggctagcctgacaggaagagctggcatcttctgaggaatgtgcaaaccatgcctgcgtctgctccatggcactagcccagtgtctgggcatttgagcagttgttctgagggctcaggatgtttatccccataagcagctgaactgcctcctgtttcgagagcagagcagaggaatgcagtggaagagacccaggcctctggcacccagattagagagttttgtgctgaggtccctatatggttgtgttagagtgaacggccagcttcagcctgtctttgctccttgtttgggaagcgagtgggaggggatcagaccagggggctatataacccttcagcattcagcctccccagacaccacccacccaga

SEQ ID NO: 6 - DNA, 142 bp, Rattus norvegicus
ggtccctatatggttgtgttagagtgaacggccagcttcagcctgtctttgctccttgtttgggaagcgagtgggaggggatcagaccagggggctatataacccttcagcattcagcctccccagacaccacccacccaga

SEQ ID NO: 7 - DNA, 207 bp, Rattus norvegicus
gagcagaggaatgcagtggaagagacccaggcctctggcacccagattagagagttttgtgctgaggtccctatatggttgtgttagagtgaacggccagcttcagcctgtctttgctccttgtttgggaagcgagtgggaggggatcagaccagggggctatataacccttcagcattcagcctccccagacaccacccacccaga

SEQ ID NO: 8 - DNA, 232 bp, Rattus norvegicus
tgaactgcctcctgtttcgagagcagagcagaggaatgcagtggaagagacccaggcctctggcacccagattagagagttttgtgctgaggtccctatatggttgtgttagagtgaacggccagcttcagcctgtctttgctccttgtttgggaagcgagtgggaggggatcagaccagggggctatataacccttcagcattcagcctccccagacaccacccacccaga

SEQ ID NO: 9 - DNA, 262 bp, Rattus norvegicus
gggctcaggatgtttatccccataagcagctgaactgcctcctgtttcgagagcagagcagaggaatgcagtggaagagacccaggcctctggcacccagattagagagttttgtgctgaggtccctatatggttgtgttagagtgaacggccagcttcagcctgtctttgctccttgtttgggaagcgagtgggaggggatcagaccagggggctatataacccttcagcattcagcctccccagacaccacccacccaga

SEQ ID NO: 10 - DNA, 502 bp, Rattus norvegicus
tcctctgcagattcgcaggtgacccaagcatcttttgttataggctaccttttgcaacagtgttgccttaaagtcccagctagtcagagacaggcccttcctcatctcaagcccttagctaatggacccaaaggctagcctgacaggaagagctggcatcttctgaggaatgtgcaaaccatgcctgcgtctgctccatggcactagcccagtgtctgggcatttgagcagttgttctgagggctcaggatgtttatccccataagcagctgaactgcctcctgtttcgagagcagagcagaggaatgcagtggaagagacccaggcctctggcacccagattagagagttttgtgctgaggtccctatatggttgtgttagagtgaacggccagcttcagcctgtctttgctccttgtttgggaagcgagtgggaggggatcagaccagggggctatataacccttcagcattcagcctccccagacaccacccacccaga

SEQ ID NO: 11 - DNA, 539 bp, Rattus norvegicus
cagactacatacaatgtggcttccataaaatgatcactcctctgcagattcgcaggtgacccaagcatcttttgttataggctaccttttgcaacagtgttgccttaaagtcccagctagtcagagacaggcccttcctcatctcaagcccttagctaatggacccaaaggctagcctgacaggaagagctggcatcttctgaggaatgtgcaaaccatgcctgcgtctgctccatggcactagcccagtgtctgggcatttgagcagttgttctgagggctcaggatgtttatccccataagcagctgaactgcctcctgtttcgagagcagagcagaggaatgcagtggaagagacccaggcctctggcacccagattagagagttttgtgctgaggtccctatatggttgtgttagagtgaacggccagcttcagcctgtctttgctccttgtttgggaagcgagtgggaggggatcagaccagggggctatataacccttcagcattcagcctccccagacaccacccacccaga

SEQ ID NO: 12 - DNA, 65 bp, Rattus norvegicus
gagcagaggaatgcagtggaagagacccaggcctctggcacccagattagagagttttgtgctga

SEQ ID NO: 13 - DNA, 25 bp, Rattus norvegicus
tgaactgcctcctgtttcgagagca

SEQ ID NO: 14 - DNA, 30 bp, Rattus norvegicus
gggctcaggatgtttatccccataagcagc

SEQ ID NO: 15 - DNA, 240 bp, Rattus norvegicus
tcctctgcagattcgcaggtgacccaagcatcttttgttataggctaccttttgcaacagtgttgccttaaagtcccagctagtcagagacaggcccttcctcatctcaagcccttagctaatggacccaaaggctagcctgacaggaagagctggcatcttctgaggaatgtgcaaaccatgcctgcgtctgctccatggcactagcccagtgtctgggcatttgagcagttgttctga

SEQ ID NO: 16 - DNA, 37 bp, Rattus norvegicus
cagactacatacaatgtggcttccataaaatgatcac

SEQ ID NO: 17 - DNA, 177 bp, Rattus norvegicus
tgatatcaagggtcagcgataaaccaacaacatgcacgtggactgtacctaagggttaacgcagttacagtgattctgacttctaagttcctcttagggtaacataggctggtgaatcctgattacatacttccatatgtaatacatacagacttcattgatactacacacagactc

SEQ ID NO: 18 - DNA, 697 bp, Mus musculus
tgctgttaagggtcagtaaaaagccagcaacatgctggaatgttaagggttaaagcagttacagtgattctgacttctaagttactctttgggcaacacaggctggttaatcctcactacatacttcaattcctatttccactcacctccctcaagaacttgatttataaacagtgtgcctaccataaaatcatgactcctctgcatatttatgggtgactcgaagcatcttttgatctaggctaccttttgcaacagtgttgcttaaaaatcgcagctagtcagagacaggcccttccttatccaagtcctcagctaatggcccaaaagactagcctgacaggggctggcatcttctgaggaatgtgcaaaccgtgcctgcgtctgtcccatgacactagcccagtgtctgggcatttaagcagttgttctgagggcttaggatgtttatccccataacgagctgagctgcctcctgtttcgggagcagaacagaggaatgcagtggaagagacccacgctctggccacccagattagagagttttgtgctgaggtccctatatggttgtgttagagtgaacggccagcttcagcccgtctttgctccttgtttgggaggcgagtgggaggggatcagagcaaggggctatataacccttcagccttcagcctccccgggacaccacccacccaga

SEQ ID NO: 19 - DNA, 715 bp, Homo sapiens
tgctgctaaattgctcggtgacaaattagtagacaaagctaatgcacaaaaaaatgaatgtagttatagtaatgcagaattccaaattcctctttgtaagacataggcctgtcaaccttgtctccatacttcagttcctggtttcattgaaccaacacaaagacacaatgtataagtacaatgtagcttccataaaaacatcactccctctatgtatttatagacagctgaagaaatatctttcttctttgcatcctaccgtggtagaagggttttaaaagtccgtgctaggcagaggcagccctttctgcccctttctgttctcagtttattaggaaatggcctgaaattccagcatgatagcaagctggcatcctctgtggaatgtcaataccatgcctgcatctgcccattaccctagctcagtgtctctgggcatttctgcagttgttctgaaggcttggcgtgtttatctcccacaagcggctgaactgcctcccgtttcatgagcagaccagtggaatgcagtggaagagacccacgctccggccacccagattagagagttttgtgctgaggtccctatatggttgtgttagactgaacgacaggctcaagtctgtctttgctccttgtttgggaagcaagtgggaggagagcaggccaaggggctatataacccttcagctttcagcttccctgaacaccacccagt

SEQ ID NO: 20 - DNA, 699 bp, Gallus gallus
cttgtccacagttcctagtgctttggaaataaactgatggagacaggatattgattgtcacccattacaggctagggacaccataacaacctgttagcagaacgtttacacagccttcaaagaccctaccatgaaccctatgcaacagcaggtacttcttttagtatccccccagtgcagaccttttaagtgaatttgtggcaaaattcagtagctgtttagcttgccgaaagtattctcattgctttggtccaaatctttaactaatgcaaagtgtctccttaaaaacactttccctattacaaatgactgctctttcagttttcactctgcctcttggatgttcctgtgaaggccagggcctctctctcttgtttgaacgtgtgctcttcctgacagagggtgtctgtcccaggcacgcttttcttgctgcattttagcaagttctgcagtgtttatcttacacagctgaaagtctcctcctgtttcatgagctctgcgttggaatgcagtggaagggactgacgcctgtcgacccagattagaggtttttgtaataaggtccctatatggttttgttagagacttcggctctgtctctctcatctctgctccttgtttgggaggctggtgggaggagaagagctgaaggggctatataaccctggtgcttttggatacacagtgcaccatcccaga

SEQ ID NO: 21 - DNA, 845 bp, artificial sequence
  Description of artificial sequence EGFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtgta

SEQ ID NO: 22 - DNA, 666 bp, Artificial sequence
  Description of artificial sequence Red Fluorescent Protein
aagaatgttatcaaggagttcatgaggtttaaggttcgcatggaaggaacggtcaatgggcacgagtttgaaatagaaggcgaaggagaggggaggccatacgaaggccacaataccgtaaagcttaaggtaaccaaggggggacctttgccatttgcttgggatattttgtcaccacaatttcagtatggaagcaaggtatatgtcaagcaccctgccgacataccagactataaaaagctgtcatttcctgaaggatttaaatgggaaagggtcatgaactttgaagacggtggcgtcgttactgtaacccaggattccagtttgcaggatggctgtttcatctacaaggtcaagttcattggcgtgaactttccttccgatggacctgttatgcaaaagaagacaatgggctgggaagccagcactgagcgtttgtatcctcgtgatggcgtgttgaaaggagagattcataaggctctgaagctgaaagacggtggtcattacctagttgaattcaaaagtatttacatggcaaagaagcctgtgcagctaccagggtactactatgttgactccaaactggatataacaagccacaacgaagactatacaatcgttgagcagtatgaaagaaccgagggacgccaccatctgttcctttag

SEQ ID NO: 23 - DNA, 719 bp, Artificial sequence
  Description of artificial sequence GFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacccacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagta

SEQ ID NO: 24 - DNA, 937 bp, Artificial sequence
  Description of artificial sequence Renilla Luciferase
atgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataat

SEQ ID NO: 25 - DNA, 3144 bp, Artificial sequence
  Description of artificial sequence beta Galactosidase
atgtcgtttactttgaccaacaagaacgtgattttcgttgccggtctgggaggcattggtctggacaccagcaaggagctgctcaagcgcgatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataa

SEQ ID NO: 26 - DNA, 1558 bp, Artificial sequence
  Description of artificial sequence SEAP
atgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacat

SEQ ID NO: 27 - DNA, 1651 bp, Artificial sequence
  Description of artificial sequence Luciferase Firefly
catggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtg

SEQ ID NO: 28 - DNA, 658 bp, Artificial sequence
  Description of artificial sequence CAT
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaactccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcacgatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgt

SEQ ID NO: 29 - DNA, 582 bp, Artificial sequence
  Description of artificial sequence hGH
atgttcccaactattccactgagtcgcctgttcgataacgcgatgctgcgtgcgcatcgtctgcaccaactggctttcgacacttaccaggagttcgaagaagcatacatcccgaaagaacagaaatacagcttccttcagaacccacagacctcgttgtgtttctctgaaagtatcccgaccccttctaaccgcgaagagacccagcagaaatcgaaccttgaactgcttcgtatctcgctgcttctcattcagtcgtggctggagccagtacagttcctgcgttcggttttcgcaaactcactggtatacggtgcgtctgacagtaacgtttacgacctgctgaaagaccttgaagaagggatccagaccctgatgggtcgcctggaagatggttcaccacgcactggtcagatcttcaaacagacttactccaaattcgatactaactctcataacgatgatgctctgctgaaaaactacggcctgctgtactgtttccgtaaagatatggataaagttgaaactttcctgcgtatcgttcagtgtcgttctgttgaagggtcgtgtggcttctaatag

SEQ ID NO: 30 - DNA, 119 bp, Oryctolagus cuniculus
tataaataaacccctctgctaaccatgttcatgccttcttctttttcctacagctcctgggcaacgtgctggttgttgtgctgtctcatcattttggcaaagaattcactcctcaggtg

SEQ ID NO: 31 - DNA, 802 bp, Gallus gallus
ccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggggtttatatcttcccttctctgttcctccgcagccagccatg

SEQ ID NO: 32 - DNA, 1032 bp, Bacteriophage P1
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 33 - DNA, 34 bp, Bacteriophage P1
ataacttcgtataatgtatgctatacgaagttat

SEQ ID NO: 34 - DNA, 384 bp, Streptomyces avidinii
gctgctgaagcaggtatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcacaaccgaggccaacgcctggaagtccacgctggtcggccacgacaccttcaccaaggtgaagccgtccgccgcctcc

SEQ ID NO: 35 - DNA, 42 bp, Artificial sequence
  Description of artificial sequence myc epitope
ggatccgaacaaaagctgatctcagaagaagatctatgcata

SEQ ID NO: 36 - DNA, 18 bp, Artificial sequence
  Description of artificial sequence 5xHis Sequence
catcaccatcatcattaa

SEQ ID NO: 37 - DNA, 29 bp, Artificial sequence
  Description of artificial sequence Primer
gtccttaagcttgatatcaagggtcagcg

SEQ ID NO: 38 - DNA, 29 bp, Artificial sequence
  Description of artificial sequence Primer
ggcttctgaattctgggtgggtggtgtct

SEQ ID NO: 39 - DNA, 19 bp, Artificial sequence
  Description of artificial sequence Primer
tgttctcaggctcaggatg

SEQ ID NO: 40 - DNA, 25 bp, Artificial sequence
  Description of artificial sequence Primer
ctgtttcgagagagcaaagcttagg

SEQ ID NO: 41 - DNA, 18 bp, Artificial sequence
  Description of artificial sequence Primer
gagtgaacggccagcttc